(19) United States
(12) Patent Application Publication
(10) Pub. No.: US 2006/0289011 A1
(43) Pub. Date: Dec. 28, 2006

(54) Resilient nasal intubation tube supporter

(76) Inventor: Paula Ann Helsel, Pittsburgh, PA (US)

Correspondence Address: William D. Breneman, Esq.;BRENEMAN & GEORGES, 3150 Commonwealth Avenue, Alexandria, VA, 22305, US

(21) Appl. No.: 11/166,202

(22) Filed: Jun. 27, 2005

(51) Int. Cl. A62B 9/06; A61M 5/32

(52) U.S. Cl. 128/207.17; 604/174

(57) ABSTRACT

A resilient nasal intubation tube supporter is provided having an adhesive coated base and a nasal intubation tube groove having an adhesive layer separated by a resilient foam body for anchoring a sinusoidal shaped nasal intubation tube in place for surgical procedures. The invention includes provision for length and height adjustment of the novel intubation tube holder. The novel intubation tube holder provides a cushioned pliantly resilient support for nasal intubation tubes and provides support for conduits and hoses that need to be attached during surgical procedures.

### CROSS REFERENCE TO RELATED APPLICATIONS

Not applicable.

### STATEMENT REGARDING FEDERALLY SPONSORED RESEARCH OR DEVELOPMENT

Not applicable.

### INCORPORATION-BY-REFERENCE OF MATERIAL SUBMITTED ON COMPACT DISC

Not applicable.

### REFERENCE TO A “MICROFICHE APPENDIX”

Not applicable.

### BACKGROUND OF THE INVENTION

1. Field of the Invention

The invention relates to a conduit support device used for facial surgery such as oral maxillary surgery under general anesthesia. More particularly the invention pertains to a device for resiliently and detachably anchoring a nasal intubation tube during major dental and medical surgery requiring access to the oral cavity under anesthesia.

2. Description of the Related Art Including in the U.S. Information Disclosed Under 37 CFR 1.97 and 1.98

The best known prior art in use for resiliently and detachably supporting nasal intubation tubes, which are known as nasal RAE and nasal tubes is illustrated in Prior Art FIG. 1. Support for the nasal intubation tube involves support not only of the nasal tube but also the anesthesia breathing circuits connected to the nasal intubation tube. These anesthesia breathing circuits can carry oxygen, nitrous oxide air, anesthesia and other anesthesia gases into the body during surgery. A tube is disposed within the body of the patient while the hook-shaped end of the nasal intubation tube require's anchoring to the patient and support away from the patient to hold the anesthesia breathing circuit necessary to sustain the patient during the operation.

Prior art operating rooms have typically employed a towel or pad 21 taped to the forehead 22 of a patient 24 to support nasal intubation tube 25. Nasal intubation tube 25 due to its sinusoidal configuration is designed to be inserted through the left or right nares down into the posterior pharynx to where the balloon is inflated just below the vocal chord. Due to the sinusoidal shape the nasal intubation tube requires support at a distance away from the forehead for which adjustment is made by repeatedly folding towel or pad 21 until it provides the proper height and width to support not only nasal intubation tube 25 but also the anesthesia breathing circuit tubes 27, 29 that supply air, oxygen, nitrous oxide, anesthesia gases and other life sustaining elements during the operation. The towel or pad is then taped in place to the patient's forehead.

Prior art towels and pads taped in place have the disadvantage of consuming intra-op time as well as resulting in inefficient taping of towel and pad procedures. The prior art towels and pads without repeated folding lack sufficient nasal intubation tube support for the anesthesia breathing circuits connected to the nasal intubation tube as well as slippage of the tube and undue tension on the nostril as illustrated in FIG. 1. Moreover all of these problems are more or less aggravated depending on the skill and experience of the anaesthetist personnel.

The best known patented prior art includes the Kalt, et al. U.S. Pat. No. 5,037,397 for a universal clamp and Geist U.S. Pat. No. 4,333,468 for a mesentery tube holder apparatus. These devices while useful for holding catheter tubes and flexible feeding tubes, chest tubes, dialysis tubes and other tubes for introducing or removing fluids from the body have not been utilized in operating rooms for nasal intubation tubes and nasal RAE tubes used in facial and dental surgery. Such prior art clamps and devices are substantially flat and are not of a configuration or structure sufficient to accommodate the substantially inflexible sinusoidal shape of nasal intubation tubes or the hook-shaped end. Further such prior art clamps and tube holders cannot support the anesthesia breathing circuits attached to the nasal intubation tube during surgical procedures without expenditure of further intra-op time or injury during surgical procedures.

Other known prior art medical conduit devices include Vergano, et al. U.S. Pat. No. 5,709,665; Muller U.S. Pat. No. 2,727,512; Sanomieri U.S. Pat. No. 3,630,195; Tollini U.S. Pat. No. 6,827,706; Abel U.S. Pat. No. 5,163,914; Bierman, et al. U.S. Pat. No. 5,833,663 and Buttaravoli U.S. Pat. No. 3,918,446. These prior art conduit restraining devices for holding flexible tubing and conduits are not suitable for nasal intubation tubes or for supporting nasal intubation tubes during surgical procedures. Of the foregoing prior art only Bierman, et al. U.S. Pat. No. 5,833,633 pertains to a conduit holder for a naso tube but Bierman, et al. U.S. Pat. No. 5,833,633 is for a naso gastric tube which is more flexible and substantially different than the nasal intubation tube in configuration, flexibility and function. As such these prior art devices are not suitable for general anesthesia surgical procedures required for reconstructive surgery, jaw fractures or full and partial dental extractions and other surgical procedures requiring nasal intubation.

### BRIEF SUMMARY OF THE INVENTION

A resilient nasal intubation tube supporter is provided for operating room procedures necessitating the use of a nasal intubation tube. The nasal intubation tube supporter accommodates the sinusoidal shaped intubation tube as well as the hook-shaped proximal end and associated anesthesia breathing circuits that are subsequently connected to the nasal intubation tube.

The novel intubation tube supporter includes a resilient and flexibly supportive body such as a sponge or cellular body having at the top surface a groove and a semi circular adhesive in the groove covered with a protective release strip. The bottom surface of the resilient and flexibly supportive body includes a substantially flat adhesive coated surface covered with a protective release strip.

The entire intubation tube supporter is extremely lightweight and unobtrusive in use and operation. The novel intubation tube supporter requires only the removal of the bottom protective release strip and placement of the intubation tube supporter on the forehead. The protective release strip is removed from the semi circular opening or groove and the nasal intubation tube is placed in the adhesive covered opening. The associated anesthesia breathing circuits may then be added to the nasal intubation tube which is both cushioned resiliently anchored and supported by the novel nasal intubation tube holder.

The novel intubation tube holder is preferably of a generally V-shape with a groove or semi circular opening disposed down the center of the V-shape. Adjustability for sizing of the novel intubation tube holder may be provided either horizontally, vertically or both. Adjustability along the vertical axis may be provided by perforating or by joining two smaller sections together medially by adhesive backing with a protective release strip holding both adhesive backed sections together. In this manner the two V-shaped sections can be rapidly separated from each other in the operating room to accommodate different forehead sizes.

The novel intubation tube holder may also be rapidly adjusted in the operating room environment for height along the horizontal axis by perforating or by joining two smaller V-shaped sections, the top one having the nasal intubation adhesive covered groove or a semi circular adhesive opening and the bottom one having the substantially flat adhesive coated surface. In embodiments where height adjustability is provided both sides of the perforated or layered sections preferably include an adhesive and where two separate sections are employed as opposed to perforations a double-backed protective release strip may be employed to hold both sections together. Separation of the two sections may then be made in the operation room rapidly and efficiently to accommodate differences in height which had heretofore been accomplished by increasing or decreasing the number of folds in the pad or towel.

Vertical and horizontal adjustability is accommodated by a combination of perforations and adhesive backing with a protective release strip holding both adhesive backed sections together. In the preferred embodiment of vertical and horizontal adjustability vertical adjustability is achieved by perforations disposed vertical to the horizontal plane of the adhesive coated based of the novel intubation tube holder. Horizontal adjustability is achieved by holding the two V-shaped sections held together having an adhesive held together by a protective release strip having a matching perforation or by two laterally adjacent release strips on either side or bridging the perforation. In this manner the length of the novel intubation tube supporter may be quickly separated by the vertical perforations to accommodate different sized foreheads and the thickness may be quickly adjusted to desired height requirements.

Other novel aspects of the invention and embodiments of the invention will be understood in conjunction with the following detailed description of the invention and the drawing(s).

### BRIEF DESCRIPTION OF THE SEVERAL VIEWS OF THE DRAWINGS

The invention will be described further in conjunction with the following drawings illustrating the best known prior art (FIG. 1) with the remaining drawings illustrating preferred embodiments and best mode of the invention in which:

FIG. 1 is a side elevational view of a prior art nasal intubation tube holder;

FIG. 2 is a side elevational view of a novel nasal intubation tube holder in accordance with the invention;

FIG. 3 is a side elevational view partly in section illustrating the novel nasal intubation tube holder holding a nasal intubation tube;

FIG. 4 is a perspective view of the novel intubation tube holder;

FIG. 5 is a side elevation view of an alternative embodiment of the novel nasal intubation tube holder;

FIG. 6 is a side view of an alternative embodiment of the novel nasal intubation tube holder;

FIG. 7 is a front end view of the novel nasal intubation tube holder;

FIG. 8 is a front end view similar to FIG. 7 of a further embodiment of the novel nasal intubation tube holder;

FIG. 9 is a front end view similar to FIG. 7 of a further embodiment of the novel nasal intubation tube holder;

FIG. 10 is a side elevational view of a further embodiment of the novel intubation tube holder illustrating vertical separation for accommodating different lengths; and

FIG. 11 is a side view of a further embodiment of the novel nasal intubation tube holder illustrating horizontal and vertical separation for accommodating different heights and lengths.

### DETAILED DESCRIPTION OF THE INVENTION INCLUDING BEST MODE

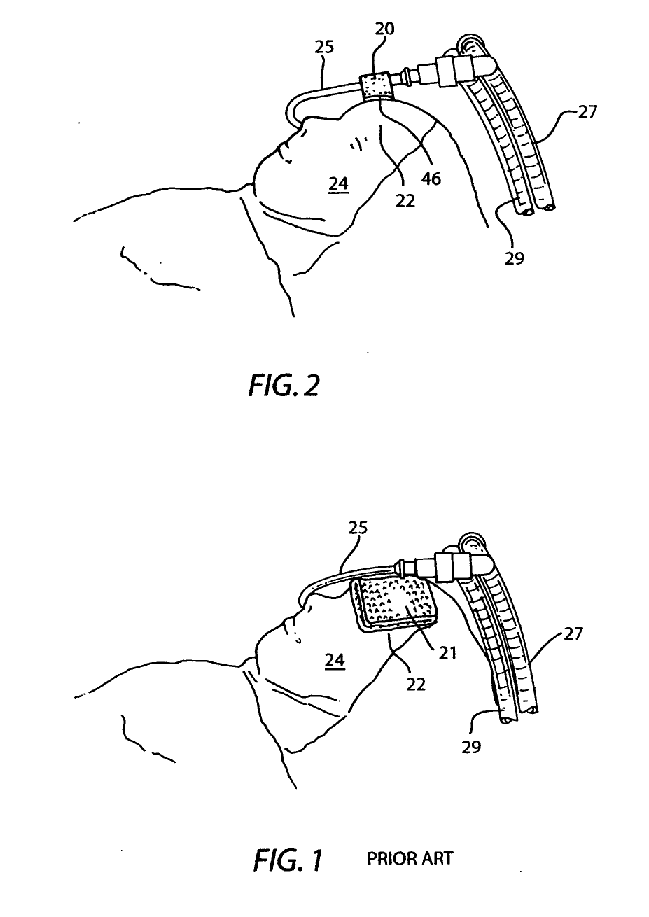

Prior art FIG. 1 illustrates the best known prior art currently employed in operating rooms for supporting nasal intubation tubes. As heretofore described the prior art employs a towel or pad 21 folded to provide the desired length, width and height to support a nasal intubation tube 25 along with the anesthesia breathing circuits having tubes 27, 29 that are connected to the nasal intubation tube 25. Towel or pad 21 is typically taped to the forehead 22 of the patient or around the patient's head or hair.

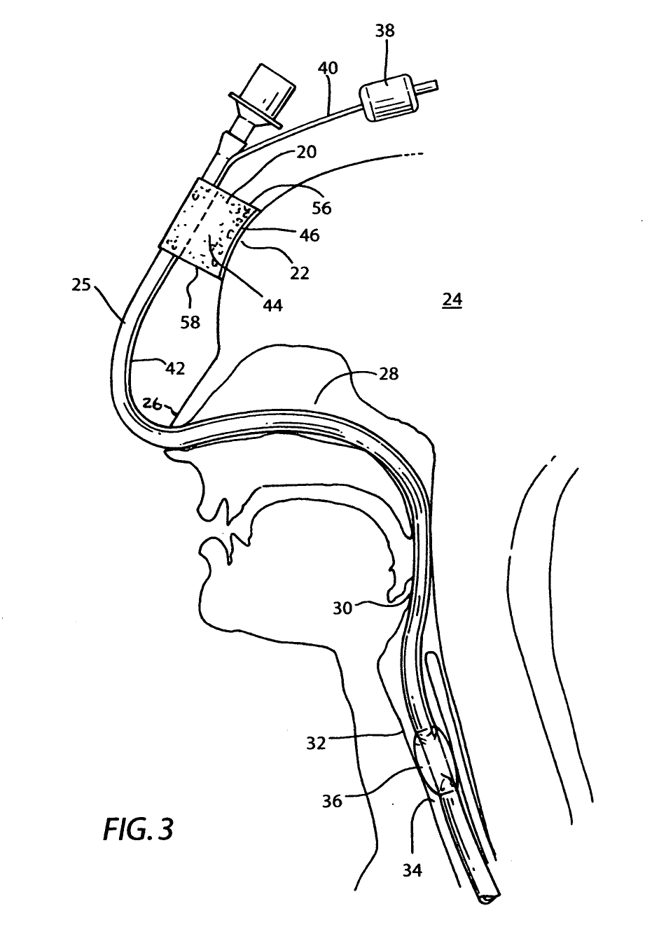

Referring now to FIG. 2, 3, 4 and 7 the novel intubation tube holder 20 constructed in accordance with the best mode and preferred embodiment is illustrated. Nasal intubation tube holder is designed to fix the position of the sinusoidal shaped nasal intubation tube 25 onto the forehead 22 of patient 24 at an elevation sufficient to accommodate the sinusoidal shaped tube which extends through nose 26 into the nasal septum 28 past the epiglottis 30 into the trachea 32 where below the vocal cords and above the chorina 34 the balloon 36 on the nasal intubation tube 25 is inflated. The balloon 36 seals off the trachea and lungs from any fluid from the esophagus and stomach and surgical field blood and irrigation. Balloon 36 is inflated through a syringe fitting 38 (FIG. 3) connected by tube 40 which connects to an internal conduit 42 in nasal intubation tube 25 to balloon 36.

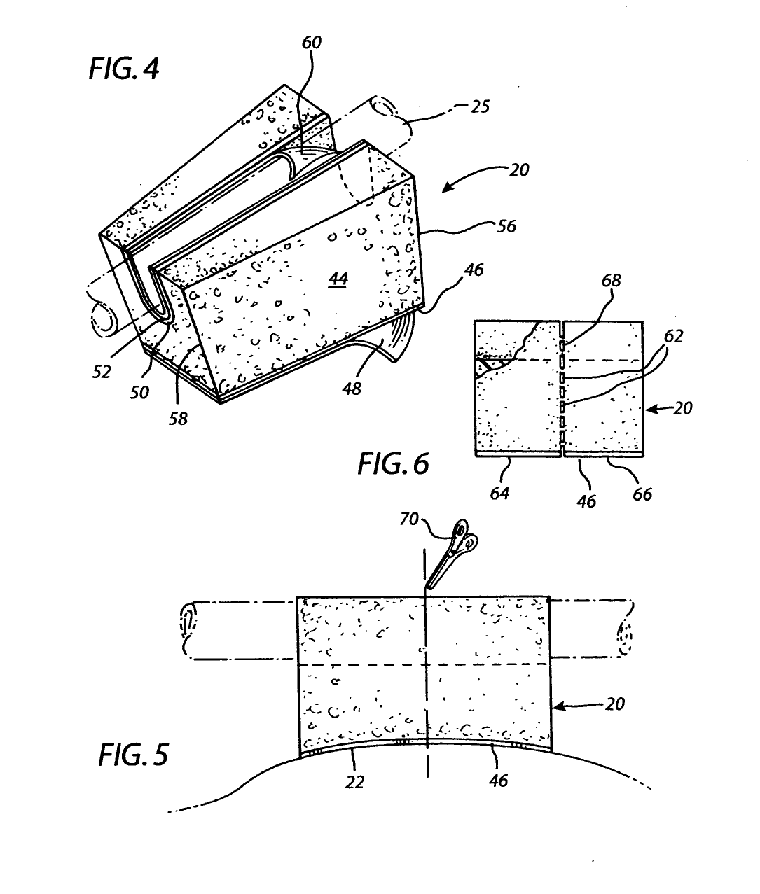

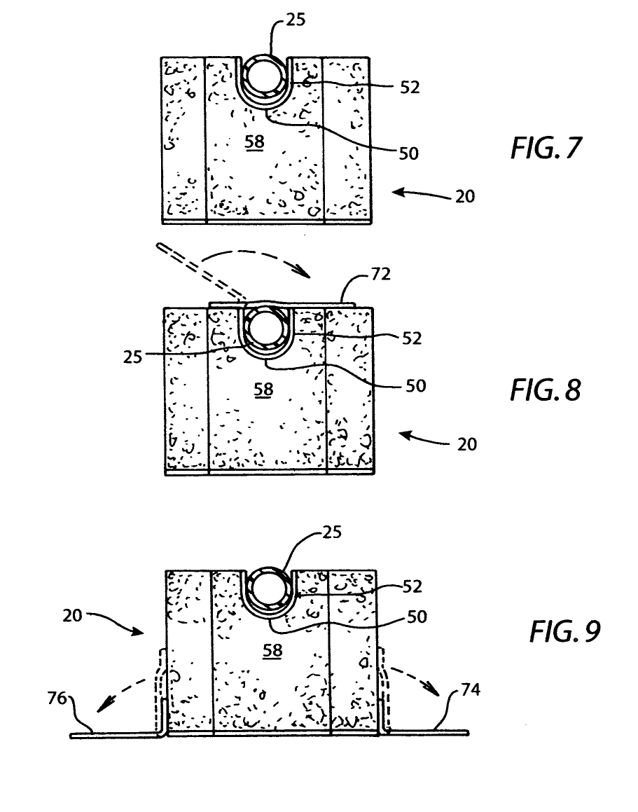

Due to the sinusoidal shape of nasal intubation tube 25 a height of about ½ inch to 2 inches is preferable to resiliently anchor the nasal intubation tube in place to prevent movement of the nasal intubation tube during surgical procedures. Anchoring of the novel nasal intubation tube holder 20 to the forehead 22 of patient 24 is both accommodated and facilitated by a firm but pliantly resilient body 44 composed of open cell or closed cell material and preferably of an elastomeric composition having a surgical tape base 46. Surgical tape base 46 is covered by protective release strip 48 (FIG. 4).

Nasal intubation tube 25 is similarly anchored in a groove or semi circular opening 50 having a surgical adhesive covered channel 52. Surgical adhesive covered channel 52 is similarly covered with a protective release strip 60 that is removed to firmly but pliantly anchor nasal intubation tube 25 to the nasal intubation tube holder. As used herein firmly but pliantly anchored means forces exerted on the nasal intubation tube are absorbed by the open or closed cell material and not transmitted to the surgical tape base 46 and movement of the forehead does not exert forces to the nasal intubation tube.

The novel intubation tube holder 20 is preferably of a tapered rectangular configuration having a greater width at its back end 56 than its front end 58. The greater width at back end 56 is designed to support anaesthetic breathing circuits having tubes 27, 29, while the narrower front end 58 is designed to center and receive the distal portion of the nasal intubation tube 25. In a further embodiment of the invention the height at the back end 56 may be greater than the height at the front end 58 to resiliently support the greater weight of the anesthesia breathing circuits. Typically the height at front end 58 is about 0.5 in to 2.0 in (1.6-5.2 cm) with a width of about 0.75 in to 2.0 in (2.4-5.2 cm) and the height at back end 56 is about 0.75 in to 3.0 in (2.4 cm to 7.6 cm) with a distance of about 1 inch to 4 inches (2.5 cm to 10.2 cm).

The novel intubation tube holder is preferably constructed of sterile or sterilizable surgical grade elastomeric foam material such as polyethylene cross linked foam, a polyvinyl chloride foam or other such foams employed in the medical arts. Medical grade adhesive or double sided medical adhesive tape can be employed on surgical tape base 46. Medical grade adhesive such as a porous acrylic copolymer adhere to both polyethylene cross linked foam as well as nasal intubation tube 25. Other suitable tapes or adhesives applied directly to an open or closed cell foam may be applied as is known to those skilled in the art to affix the novel intubation tube holder 25 to a patient's skin without irritation. In either application of the invention of whether a double backed surgical tape or adhesive is applied to the elastomeric foam a protective release strip 48 or 60 is utilized to preserve the adhesive until use.

Referring now to FIGS. 5 and 6 an alternative embodiment or application of the novel nasal intubation tube holder 20 is illustrated. FIG. 6 illustrates an embodiment of the invention providing for the vertical separation of the tube holder 20 by a pre perforated separation line 62. In this embodiment the surgical tape base 46 is divided into two separate sections 64 and 66 to allow the novel nasal intubation tube holder to be pulled apart to accommodate a smaller forehead. Similarly surgically adhesive covered channel 52 may also include perforations 68 in the tape or protective release strip 60 to assist in the quick separation of the two sections of the novel intubation tube holder. Alternatively the novel intubation tube holder 20 may be cut to size with scissors 70 before it is applied to the patient's forehead 22.

Referring now to FIGS. 8 and 9 further embodiments of the invention are illustrated. In FIG. 8 novel intubation tube holder 20 includes an optional flap 72 for further restraining nasal intubation tube 25 in place. In FIG. 9 optional or additional side adhesive tape tabs 74 and 76 are illustrated. Optional or additional side adhesive tape tabs 74 and 76 may be added or used depending upon the desires of the surgical anesthesia team. Optional side adhesive tape tabs 74 and 76 must be used where horizontal separation to accommodate height is accomplished with horizontal perforations that do not include adhesive coated sections held together centrally by a release strip.

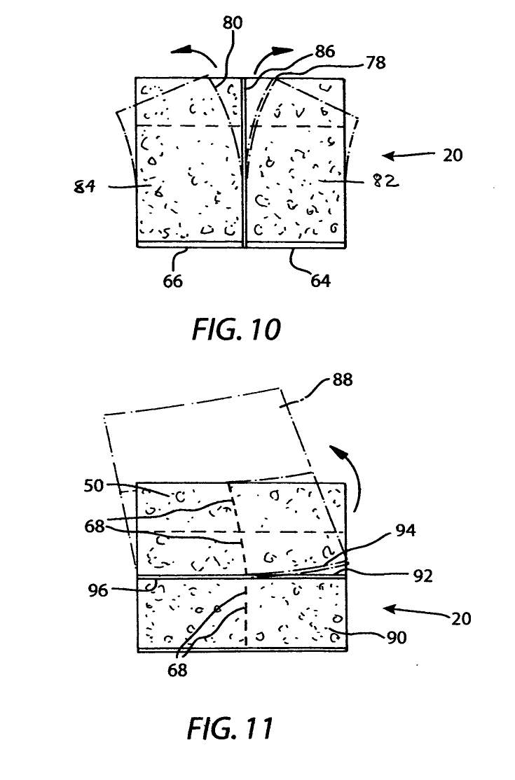

Referring now to FIG. 10 further modification of the novel nasal intubation tube holder 20 is illustrated. In FIG. 20 vertical separation is provided by having adhesive 78 and 80 applied to two sections 82 and 84 which are held together by a release strip 86. The two sections 82 and 84 may be of substantially the same length as shown or be of different lengths. Release strip is provided to stay with the section of the novel intubation tube holder being used before one of the protective release strip tapes are removed from separate sections 64 and 66 for application to the patient's forehead.

A further embodiment of the invention is provided in FIG. 11 which provides for both horizontal and vertical adjustment of the novel nasal intubation tube holder. Horizontal separation for height is provided in this embodiment by having sections 88 and 90 held together by a release strip 92. Section 88 includes channel 50 and its own adhesive base 94 which is then separated from release strip 92 and attached to the patient's forehead to accommodate variations in height. In addition a separation line 96 may either divide sections 88 and 90 down the center or be angled to provide for sections of a different size or of a complimentary geometrical configuration while accommodating differences in height engagement along the section of nasal intubation tube supporter. In addition differences length requirements may be accommodated by perforations 68 disposed through sections 88 and 90 to accommodate different requirements for length. Perforations 68 in FIG. 10 and FIG. 6 may be down the middle of the novel intubation tube holder or offset from the middle toward the front end or back end to provide nasal intubation tube holders of different lengths.

Other advantages, modifications and applications will occur to those skilled in the art. For example changes may be made in the geometrical configuration by modifying the generally tapered rectangular configuration to a frustro conical shape with a flat base. In addition modifications may be made by those skilled in the art by providing more than one nasal intubation tube channel in one or more sections of the novel nasal intubation tube holder. These and other such modifications by those skilled in the art are deemed to be included in the following claims.

As used herein and in the following claims, the word ‘comprising’ or ‘comprises’ is used in its technical sense to mean the enumerated elements include but do not exclude additional elements which may or may not be specifically included in the dependent claims. It will be understood such additions, whether or not included in the dependent claims, are modifications that both can be made within the scope of the invention. It will be appreciated by those skilled in the art that a wide range of changes and modification can be made to the invention without departing from the spirit and scope of the invention as defined in the following claims:

1. A device for holding nasal intubation tubes comprising:
(a) a sterile elastomeric foam body having a substantially flat base and an upper support surface distanced from said substantially flat base by said elastomeric foam body;
(b) an adhesive securing material substantially covering said substantially flat base;
(c) a protective release strip covering said adhesive securing material;
(d) a nasal intubation tube groove disposed laterally across said upper support surface said nasal intubation tube groove having a front end and a back end;
(e) an adhesive disposed in said nasal intubation tube groove; and
(f) a protective release strip covering said adhesive disposed in said nasal intubation tube groove.

2. The device of claim 1 further comprising means for adjusting the length of said sterile elastomeric foam body.

3. The device of claim 2 wherein said means for adjusting the length of said sterile elastomeric foam body is a plurality of perforations vertically disposed to said substantially flat base.

4. The device of claim 1 wherein said adhesive securing material substantially covering said substantially flat base and said adhesive disposed in said nasal intubation tube groove is a double backed surgical tape.

5. The device of claim 1 further comprising means for adjusting the height of said sterile elastomeric foam body.

6. The device of claim 5 wherein said means for adjusting the height of said sterile elastomeric foam body is a separation protective release strip separating adhesive coated sections of said sterile elastomeric foam body.

7. The device of claim 6 wherein the distance between said separation protective release strip to said nasal intubation tube groove is closer vertically to said front end than to said back end.

8. The device of claim 5 further comprising perforations in said sterile elastomeric foam body for adjusting length.

9. The device of claim 1 further comprising a flap for covering said nasal intubation tube groove.

10. The device of claim 1 further comprising surgical tape tabs disposed on at least one side of said sterile elastomeric foam body.

11. A nasal tube holder comprising:
(a) a resiliently compressible foam body having a substantially flat base and an upper surface distanced at from about 0.5 inches to about 3.0 inches from said substantially flat base;
(b) a nasal tube groove disposed in said upper surface having an adhesive and a protective release strip covering said adhesive;
(c) an adhesive disposed on said substantially flat base; and
(d) a protective release strip covering said adhesive disposed on said substantially flat base.

12. The nasal tube holder of claim 11 further comprising perforations disposed substantially perpendicular to said substantially flat base.

13. The nasal intubation tube holder of claim 11 wherein said adhesive on said substantially flat base is a double sided adhesive coated tape.

14. The nasal intubation tube holder of claim 11 wherein sides of said resiliently compressible foam body include surgical tape tabs.

15. The nasal intubation tube holder of claim 11 wherein said nasal tube groove is disposed at an angle to said substantially flat base.

16. The nasal intubation tube holder of claim 11 wherein said resiliently compressible foam body tapers from one end to the other.

17. A device for supporting a nasal intubation tube comprising:
(a) a resilient foam body having an upper support surface, a front end, a back end and a substantially flat base;
(b) a first adhesive material substantially covering said substantially flat base;
(c) a protective release strip covering said first adhesive material;
(d) a nasal intubation tube groove disposed across said upper surface from said front end to said back end;
(e) a second adhesive disposed in said nasal intubation tube groove; and
(f) a protective release strip covering said second adhesive.

18. The device for supporting a nasal intubation tube of claim 17 further comprising perforations disposed through said resilient foam body substantially perpendicular to said substantially flat base.

19. The device for supporting a nasal intubation tube of claim 17 wherein said first adhesive and said second adhesive are double backed surgical tape.

20. The device for supporting a nasal intubation tube of claim 17 wherein said back end is wider than said front end.
